(19) United States
(12) Patent Application Publication
(10) Pub. No.: US 2024/0099939 A1
(43) Pub. Date: Mar. 28, 2024

(54) SAFETY ASSEMBLY FOR THE RECONSTITUTION, THE TAKING AND THE INFUSION OF PHARMACOLOGICAL LIQUIDS

(71) Applicant: NACATUR INTERNATIONAL IMPORT EXPORT S.R.L., Monte Porzio (PU) (IT)

(72) Inventor: Stefana NAVARRA, Monte Porzio (IT)

(21) Appl. No.: 17/768,997

(22) PCT Filed: Oct. 21, 2020
(86) PCT No.: PCT/EP2020/079661

§ 371 Date: Apr. 14, 2022

(30) Foreign Application Priority Data
Oct. 31, 2019 (IT) 102019000020206

(51) Int. Cl. A61J 1/20; A61J 1/14

(52) U.S. Cl. CPC A61J 1/2096; A61J 1/1481

(57) ABSTRACT

A safety assembly having a syringe with a coupler, a user device with a coupler and a safety device with a first coupler configured in such a way to be coupled with the coupler of the syringe, and a second coupler configured in such a way to be coupled with the coupler of the user device. The safety device also has an axial duct and a first and a second membrane disposed inside the axial duct, each membrane being in a closed position and in an open position. The first membrane is in an open position when the coupler of the syringe is coupled with the first coupler. The second membrane is in an open position when the coupler of the user device is coupled with the second coupler.

The present invention relates to a safety assembly for the reconstitution, the taking and the infusion of pharmacological liquids, in particular antiblastic and/or hazardous medicines. The sector of reference is health safety, in particular the safety of health operators and patients during the preparation, the administration or the manipulation of potentially toxic pharmacological liquids.

Antiblastic and/toxic medicines are known, which are potentially hazardous for human health, for the patients and for the health operators (chemists, doctors and nurses) who manipulate said medicines on a daily basis. Health operators and patients suffer a severe risk of accidental exposure to antiblastic medicines during preparation and administration.

Generally speaking, antiblastic medicines for intravenous use need to be reconstituted, if they are lyophilized, or diluted, if they are in a concentrated solution, or taken from the bottle, if they are ready, and infused by means of couplings to access points along infusion lines.

In particular, the medicine is sucked with a syringe and is introduced in a bag of physiological saline or extemporaneous bolus in order to be administered to the patient. In general, medicines are transferred from the bottle to the infusion line by means of needle-less syringes connected with connection devices provided with first coupling means for coupling with the syringe and second coupling means for coupling with the preparation line and finally with the infusion line.

However, when using said connection devices, the health operators who are in charge of manipulation may be accidentally exposed to the chemotherapeutical medicines because of possible leaks during the uncoupling, the transfer and the coupling of the needle-less syringe/access assembly.

Such an inconvenience may involve a contamination risk for health operators and patients both during preparation and administration.

The medicines to be administered are generally prepared in a remote place relative to the location of the patient and the infusion line; therefore, during the transportation from the place of preparation to the place of infusion, a portion of the liquid/medicine may leak from the connection device, thus exposing operators or patients to a contamination risk.

US2015/297839 and US2018/200498 disclose an adapter suitable for being connected to the tip of a needle syringe and to a patient device. A first membrane is disposed in the adapter and a second membrane in the patient device. The two membranes are closed and pierced by the needle of the syringe when the adapter is coupled with the syringe and with the patient device. Obviously, such an adapter does not work with a needle-less syringe.

U.S. Pat. No. 4,768,568 discloses a protection apparatus suitable for being coupled with an opening of a vial. Said protection apparatus has two closed membranes that are pierced by a needle of a syringe.

WO2008/153560 discloses a medical device for the supply of gas provided with a closed membrane disposed above a filter. The tip of a needle pierces the membrane in order to inject the gas in the filter disposed under the membrane.

US2011/015566 discloses an adapter for a needle-less syringe. Such an adapter comprises a first membrane with a hole, a second membrane with a hole and a cannula disposed between the two membranes. The syringe has a head that compresses the first membrane, enlarging the hole of the first membrane to let the liquid pass through, and pushes the cannula that compresses the second membrane, enlarging the hole of the second hole to let the liquid pass through. It must be noted that the adapter is not provided with coupling means for coupling with a syringe and a patient device. The forward movement of the syringe head towards the adapter causes a compression of the two membranes, piercing the holes of both membranes. Therefore, said adapter is impaired by the fact that the medicine may leak during the transportation of the syringe/adapter assembly if the syringe is accidentally actuated when the adapter is not yet coupled with a patient device.

The purpose of the present invention is to overcome the drawbacks of the prior art by disclosing an assembly for the reconstitution, the taking and the infusion of pharmacological liquids that is reliable, inexpensive and extremely safe, and prevents the liquid from leaking during the coupling or the uncoupling of the safety device with a syringe disposed upstream and with a patient device disposed downstream.

These purposes are achieved according to the invention with the characteristics of the appended independent claim 1.

Advantageous embodiments appear from the dependent claims.

The safety assembly according to the invention is defined by claim 1.

For the sake of clarity, the description of the assembly according to the invention continues with reference to the appended drawings, which have a merely illustrative, not limiting value, wherein:

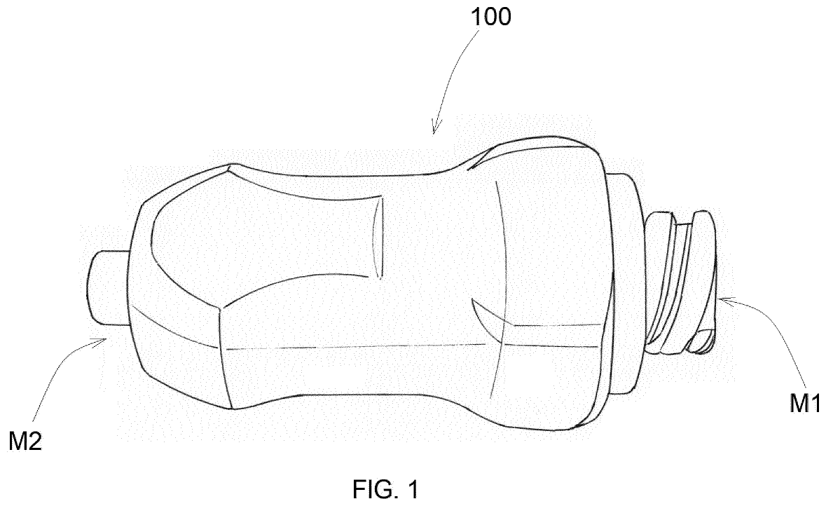

FIG. 1 is a side view of the safety device of the assembly according to the invention;

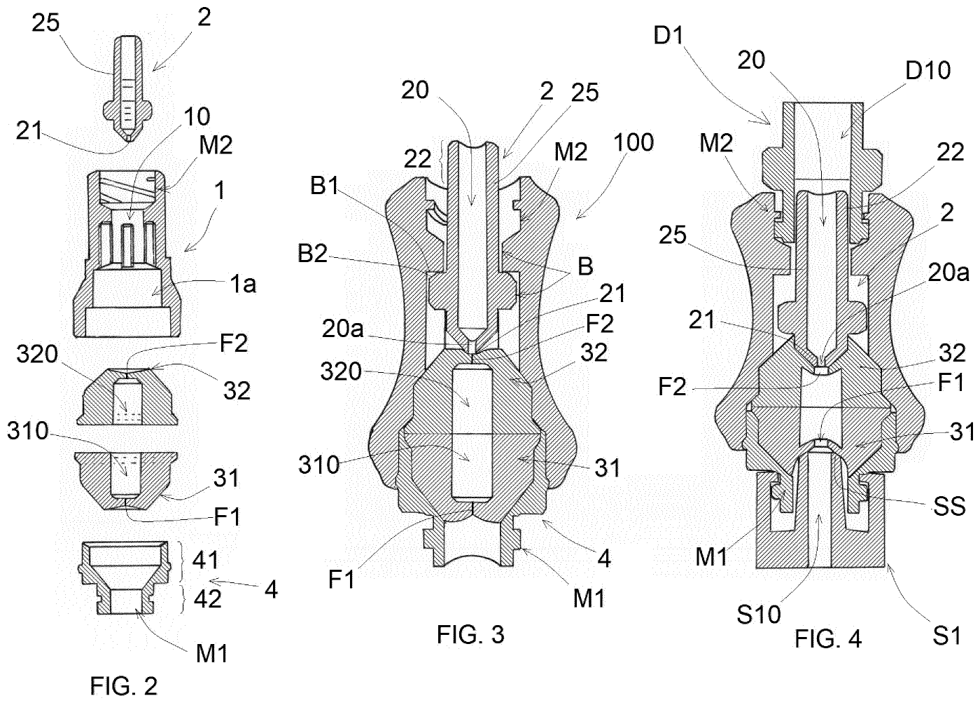

FIG. 2 is an exploded axial view of the safety device of the assembly according to the invention;

FIG. 3 is an axial view of the safety device, wherein the coupling means of the safety device are not coupled;

FIG. 4 is an axial view of the safety device, wherein the coupling means of the safety device are coupled with corresponding coupling means;

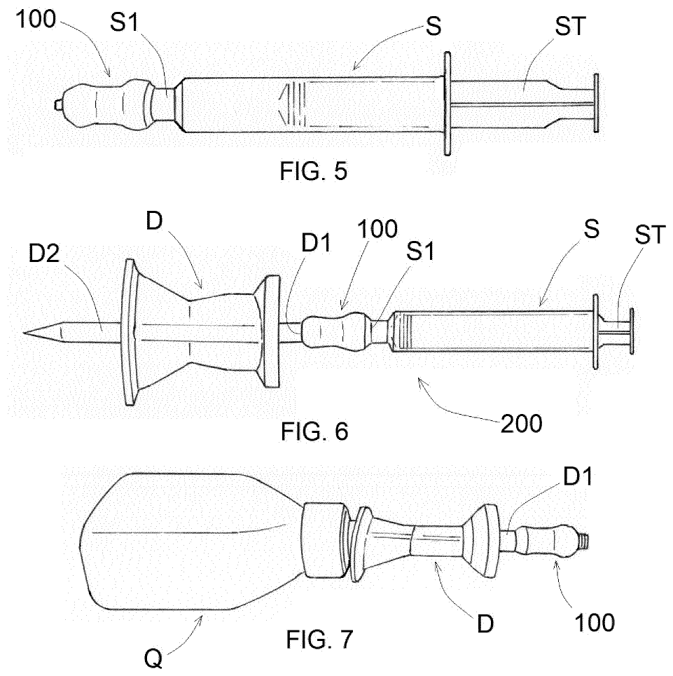

FIG. 5 is a diagrammatic side view of a needle-less syringe connected to the safety device;

FIG. 6 is a diagrammatic side view of the safety device connected to a needle-less syringe disposed upstream the safety device and to a patient device disposed downstream the safety device, wherein the patient device has a needle;

FIG. 7 is a diagrammatic side view of the safety device connected to a patient device, wherein the patient device is connected to a bottle.

With reference to FIG. 6, a safety assembly for the reconstitution, the taking and the infusion of pharmacological liquids, which is generally indicated with reference numeral (200), is disclosed. The safety assembly is suitable for being especially used with antiblastic and/or hazardous pharmacological liquids.

The safety assembly (200) comprises: a safety device (100), a syringe (S) and a user device (D). The safety device (100) is suitable for being coupled with the syringe (S) and the user device (D).

The user device (D) may have a needle (D2) for the injection of the pharmacological fluid or the user device (D) can be connected to a receptacle (Q) (FIG. 7), such for example a vial, a bottle, or a bag, for the administration/infusion of the pharmacological fluid.

With reference to FIGS. 5 and 6, the syringe (S) is a needle-less syringe. The syringe (S) has a head comprising coupling means (S1) suitable for coupling with the safety device (100).

With reference to FIG. 4, the syringe (S) comprises an axial duct (S10) with an opening obtained in correspondence of said coupling means (S1).

With reference to FIGS. 6 and 7, the user device (D) comprises coupling means (D1) suitable for coupling with the safety device (100).

With reference to FIG. 4, the user device (D) comprises an axial duct (D10) with an opening obtained in correspondence of said coupling means (D1).

The user device (D) is suitable for being hydraulically connected to the receptacle (Q) for taking or injecting a pharmacological liquid. The user device (D) may be a spike, a tube, an injection system or the like.

With reference to FIGS. 2, 3 and 4, the safety device (100) comprises a body (1) with concave external walls to favor the grip of the safety device (100).

The safety device (100) comprises:
first coupling means (M1) configured in such a way to be coupled with the coupling means (S1) of the syringe (S) in such a way that the syringe is fixed to the safety device; and
second coupling means (M2) configured in such a way to be coupled with the coupling means (D1) of the user device (D), in such a way that the user device is fixed to the safety device.

The first coupling means (M1) and the second coupling means (M2) of the safety device are disposed at opposite ends.

The safety device (100) comprises an axial duct (10) obtained along the body (1) that extends from the first coupling means (M1) to the second coupling means (M2).

When the first coupling means (M1) of the safety device (100) are coupled with the coupling means (S1) of the syringe (S), the axial duct (10) of the safety device (100) and the axial duct (S10) of the syringe are coaxial.

When the second coupling means (M2) of the safety device (100) are coupled with the coupling means (D1) of the user device (D), the axial duct (10) of the safety device (100) and the axial duct (D10) of the user device (10) are coaxial.

Advantageously, the coupling means (S1) of the syringe (S) comprise a luer-lock male connector, the coupling means (D1) of the user device (D) comprise a luer-lock female connector; the first coupling means (M1) of the safety device (100) comprise a luer-lock female connector; and the second coupling means (M2) of the safety device (100) comprise a luer-lock male connector.

With reference to FIGS. 2, 3 and 4, the safety device (100) also comprises a first membrane (31) and a second membrane (32) disposed inside the axial duct (10). Each membrane (31, 32) may be in a non-compressed closed position, wherein the membrane (31, 32) obstructs the axial duct (10), and in a compressed open position, wherein the membrane (31, 32) does not obstruct the axial duct (10).

The first membrane (31) is normally closed and may be compressed to an open position when the coupling means (S1) of the syringe (S) are coupled with the first coupling means (M1) of the safety device (100).

The second membrane (M2) is normally closed and may be compressed to an open position when the coupling means (D1) of the user device (D) are coupled with the second coupling means (M2) of the safety device (100).

The safety assembly (200) comprises:
first compression means (SS) associated with the coupling means (S1) of the syringe that compress the first membrane (31) to the compressed open position, when the coupling means (S1) of the syringe are coupled with the first coupling means (M1) of the safety device (100); and
second compression means (2) associated with said coupling means (D1) of the user device that compress the second membrane (32) to the compressed open position, when the coupling means (D1) of the user device are coupled with said second coupling means (M2) of the safety device (100).

The second compression means (2) compress the second membrane (32) in such a way to cause its deformation in the open position. The second compression means (2) comprise a cannula (25) that is mounted with possibility of axially sliding inside the axial duct (10) of the safety device (100).

The cannula (25) may slide from a non-operating position (FIG. 3), wherein the second membrane (32) is not compressed, to an operating position (FIG. 4), wherein the second membrane (32) is compressed, when said coupling means (D1) of the user device (D) are coupled with the second coupling means (M2) of the safety device (100).

The cannula (25) comprises a tip (21) that pushes the second membrane (32) to its open position, when the cannula (25) is in its operating position. The cannula (25) comprises a through axial duct (20) and a hole (20a) obtained in the tip (21).

With reference to FIG. 3, when the coupling means (D1) of the user device (D) are not yet coupled with the second coupling means (M2) of the safety device (100), the tip (21) of the cannula rests on the second membrane (32), without compression, and an ending portion (22) of the cannula externally protrudes from the axial duct (10) of the body of the safety device, in correspondence of the second coupling means (M2) of the safety device (100).

With reference to FIG. 4, when the coupling means (D1) of the user device (D) are coupled with the second coupling means (M2) of the safety device (100), the ending portion (22) of the cannula (25) is coupled by interference inside the axial duct (D10) of the user device (D). In such a way, the user device pushes the cannula (25) to its operating position towards the second membrane (32), with compression.

With reference to FIG. 4, the first compression means (SS) comprise a tip of the coupling means (S1) of the syringe (S) suitable for pushing the first membrane (31) in such a way to compress the first membrane (31) to its open position, when said coupling means (S1) of the syringe (S) are coupled with the first coupling means (M1) of the safety device (100).

With reference to FIGS. 3 and 4, each membrane (31, 32) comprises a central hole (F1, F2) that, when said membrane (31, 32) is in closed position, is suitably dimensioned to prevent a passage of a liquid and therefore the axial duct (10) of the safety device is obstructed. On the contrary, when the membrane (31, 32) is disposed in said open position, said central hole (F1, F2) is enlarged, generating a through opening that permits the passage of a liquid, as shown in FIG. 4.

With reference to FIGS. 2, 3 and 4, the safety device (100) is provided along said axial duct (10) with a seat (1a) suitable for receiving the two membranes (31, 32).

The two membranes (31, 32) are identical and opposite. More precisely, the two membranes (31, 32) are shaped like a semi-body disposed in mutual contact, and provided with ducts (310, 320) in coaxial position and in communication with the holes (F1, F2). The duct (310, 320) of each membrane (31, 32) ends in the duct (320, 310) of the other membrane (32, 31) on one side, and in the hole (F1, F2) of the membrane (31, 32) on the other side.

The safety device (100) also comprises a lid (4) that comprises a first portion (41) inserted in the axial duct (10) of the safety device (100), and a second portion (42) that comprises said first coupling means (M1) of the safety device (100).

The seat (1a) that houses the two membranes (31, 32) is defined by the first portion (41) of the lid (4) and by a portion of the axial duct (10) of the safety device (100).

Preferably, the second membrane (32) constantly pushes the cannula (25) to its non-operating position. More precisely, the second membrane (32) comprises elastic elements that tend to bring the second membrane (32) to its closed position, consequently pushing the cannula (25) to its non-operating position. So, the second membrane (32) acts a pushing means for the cannula (25). Alternatively, the safety device (100) may comprise pushing means to push the cannula (25) to its non-operating position. For illustrative purposes, said pushing means may consist in a helical spring disposed around the cannula (25) to push the cannula to its non-operating position.

With reference to FIGS. 3 and 4, the safety device (100) comprises stop means (B) suitable for stopping said cannula (25) in its non-operating position to prevent the cannula from completely coming out from the axial duct (10) of the safety device (100). The stop means (B) comprise a stop tooth (B1), which is preferably shaped like an annular collar obtained in the longitudinal duct (10) that protrudes radially outwards, and a plurality of radial tabs (B2) obtained around said cannula that protrude radially outwards. The radial tabs (B2) are suitable for being stopped against the stop tooth (B1) when the cannula (25) is in non-operating position.

Following is a description of the use and the operation of the safety device (100).

Firstly, a needle-less syringe (S) with coupling means (S1) is provided, which is connected to the safety device (100) with the first coupling means (M1), as shown in FIG. 5. In view of the above, the first compression means (SS) of the coupling means (S1) of the syringe (S) push the first membrane (31) to its open position, thus freeing the passage through the first membrane (31).

Successively, the second coupling means (M2) of the safety device (100) are connected to the coupling means (D1) (FIG. 6) of a user device (D) disposed downstream the safety device (100) and connected to the bottle, the vial or the bag. In view of the above, the cannula (25) is disposed in operating position, and the second membrane (32) is pushed by the cannula (25) to its open position, thus freeing its passage. In such a case, the two membranes (31, 32) are in open position (FIG. 4) and therefore, by pulling a plunger (ST) of the syringe (S) backwards, it is possible to take the liquid contained inside the bottle or the vial or the bag connected to said user device (D).

After taking the liquid, which is introduced in the syringe (S), the safety device (100) is disconnected from the user device (D) and, consequently, the cannula (25) is disposed in non-operating position, and the second membrane (32) is disposed in closed position, so that the liquid is prevented from leaking from the duct (10) of the safety device (100).

In such a way, the syringe (S) and the safety device (100), which are connected one to another, can be transported from the point of taking/preparation to a point of administration/infusion in a safe way.

After transporting the syringe (S) and the safety device (100) to the point of administration/infusion, the second coupling means (M2) of the safety device (100) are coupled with coupling means (D1) of another user device (D) connected to a receptacle (Q) (FIG. 7) with the pharmacological fluid to be administered/infused.

The coupling of the second coupling means (M2) of the safety device (100) with the coupling means (D1) of the user device (D) disposes the cannula (25) in operating position, consequently positioning the second membrane (32) in open position. In view of the above, the syringe and the user device (D) are hydraulically connected by means of the safety device and, when pushing the plunger (ST) of the syringe, the liquid contained in the syringe (S) will pass in the axial duct (10) of the safety device, then in the axial duct (D10) of the user device (D), being finally poured in the receptacle (Q) connected to the user device (D).

After emptying the syringe (S), the syringe (S) can be uncoupled from the safety device (100) (see FIG. 7) by uncoupling the coupling means (S1) of the syringe (S) and the first coupling means (M1) of the safety device (100).

In such a case, the first membrane (31) is disposed in closed position, closing the passage and preventing the liquid from leaking from the axial duct (10) of the safety device (100).

The advantages of the present invention are manifest after the preceding description.

In particular, the safety device (100) generates a double safety in both flowing directions of the liquid. In fact, the safety device (100) permits the passage of liquid inside the axial duct (10), during the taking and during the administration, only if a syringe (S) and the user device (D) are coupled with the safety device (100).

When the safety device (100) is not connected with an element disposed upstream or downstream, i.e. when the coupling means (M1, M2) of the safety device (100) are not connected with coupling means (S1, D1) of another element, then the liquid is prevented from leaking because one of the two membranes (31, 32), which act as safety valves, is closed.

Therefore, the liquid (chemotherapeutic medicine or toxic liquids) will not leak from the axial duct (10) in case of uncoupling of the coupling means (M1, M2) because of the fact that, also in this condition, a tight compartment is determined by the closure of one of said two membranes (31, 32).

In conclusion, the safety device (100) permits to transport syringes and injection systems in a very safe way, avoiding leaks of hazardous medicine from the syringe or from the injection system that may be dispersed in the environment or go in direct contact with a patient or with an operator with severe consequences for their health.

Although the coupling means (M1, M2, S1, D1) illustrated in the appended figures comprise dual-lock connectors. said coupling means may comprise magnetic coupling means, bayonet coupling means or the like.

1. Safety assembly for the reconstitution, the taking or the infusion of pharmacological liquids, comprising:
a needle-less syringe, comprising an axial duct in communication with an opening, and coupling means in correspondence of said opening;
a user device comprising an axial duct in communication with an opening and coupling means in correspondence of said opening, and
a safety device comprising:
first coupling means configured in such a way to be coupled with said coupling means of the syringe in such a way to fix the safety device to the syringe,
second coupling means configured in such a way to be coupled with the coupling means of the user device in such a way to fix the safety device to the user device,
an axial duct that extends from the first coupling means to the second coupling means, and
a first and a second membrane disposed inside said axial duct; each membrane being provided with a through hole and being in a non-compressed closed position wherein the hole of the membrane is closed and obstructs the passage of liquid in the axial duct, and in a compressed open position, wherein the hole of the membrane is open and does not obstruct the passage of liquid in the axial duct (10);
said safety assembly also comprises:
first compression means associated with said coupling means of the syringe to compress the first membrane to the compressed open position, when the coupling means of the syringe are coupled with said first coupling means of the safety device; and
second compression means associated with said coupling means of the user device to compress the second membrane in the compressed open position, when the coupling means of the user device are coupled with said second coupling means of the safety device.

2. The safety assembly of claim 1, wherein said coupling means of the syringe comprise a male luer-lock connector; said coupling means of the user device comprise a female luer-lock connector; said first coupling means of the safety device comprise a female luer-lock attachment; said second coupling means of the safety device comprise a male luer-lock connector.

3. The safety assembly of claim 1, wherein said second compression means comprise a cannula that is slidingly inserted in the axial duct of the safety device; said cannula being suitable for sliding from a non-operating idle position, wherein said second membrane is not compressed, to an operating position, wherein said second membrane is compressed; said cannula comprising an end portion suitable for being coupled inside the axial duct of the user device, and a tip suitable for pushing said second membrane to its compressed open position, when said coupling means of the user device are coupled with the second coupling means of the safety device.

4. The safety assembly of claim 1, wherein said first compression means comprise a tip of the coupling means of the syringe suitable for pushing said first membrane to the open position, when said coupling means of the syringe are coupled with said second coupling means of the safety device.

5. The safety assembly of claim 1, wherein said membranes are shaped like a semi-body with a channel in communication with said hole and are disposed in opposite position in mutual contact.

6. The safety assembly of claim 1, wherein said safety device is provided along said axial duct with a seat for said two membranes.

7. The safety assembly of claim 1, wherein said safety device comprises a lid with a first portion inserted in the axial duct of the safety device, and a second portion comprising said first coupling means of the safety device.

8. (canceled)

9. The assembly of claim 3, wherein said second membrane pushes said cannula to its non-operating position.

10. The assembly of claim 3, wherein said safety device comprises stop means suitable for stopping said cannula in its non-operating position; said stop means comprising at least one stop tooth obtained in the longitudinal duct and a radial tab obtained in said cannula, said radial tab being suitable for being stopped against said stop tooth when said cannula is in said non-operating position.

11. The safety assembly claim 6, wherein said safety device comprises a lid with a first portion inserted in the axial duct of the safety device, and a second portion comprising said first coupling means of the safety device.

12. The assembly of claim 11, wherein said seat that houses the two membranes is defined by the first portion of the lid and by a portion of the axial duct of the safety device.
